(19) United States
(12) Patent Application Publication
(10) Pub. No.: US 2026/0102253 A1
(43) Pub. Date: Apr. 16, 2026

(54) MOUNTING BOSS FOR ATTACHING AN INTRAMEDULLARY COMPONENT TO AN ARTIFICIAL JOINT

(71) Applicant: ORTHO DEVELOPMENT CORPORATION, Draper, UT (US)

(72) Inventors: Michael D. Ensign, Salt Lake City, UT (US); Bao-Khang Ngoc Nguyen, Holladay, UT (US)

(73) Assignee: ORTHO DEVELOPMENT CORPORATION, Draper, UT (US)

(21) Appl. No.: 18/911,915

(22) Filed: Oct. 10, 2024

(51) Int. Cl. A61F 2/38; A61F 2/46

(52) U.S. Cl. CPC A61F 2/3886; A61F 2/3859; A61F 2/461; A61F 2/4637

(57) ABSTRACT

A mounting boss for attaching an intramedullary bone attachment component such as a sleeve or stem to an artificial joint component is provided. The mounting boss includes a mounting feature which deforms the mounting surface on the joint component to prevent undesired movement of the mounting boss after assembly to the joint component.

### THE FIELD OF THE INVENTION

The present invention relates to prosthetic joints. In particular, examples of the present invention relate to a mounting boss for the femoral component of a prosthetic knee joint that may be used to attach a bone ingrowth sleeve or stem to the femoral component.

### INTRODUCTION

In repairing a damaged joint, modular joint prostheses allow surgeons to create a larger number of joint configurations while maintaining a reduced inventory of joint components. In repairing a knee joint, it may be desirable to attach a bone ingrowth sleeve or a stem to a femoral component to create a more secure attachment of the prothesis to the femur. Use of a modular mounting boss to attach a sleeve or stem to a femoral component allows a surgeon to vary the angle or position of the sleeve or stem relative to the femoral component to best fit the sleeve or stem to anatomy of the patient femur. Modular attachment of the sleeve or stem to the femoral component allows a single femoral component, a single sleeve or stem, and a few mounting bosses to accommodate a wide variation of patient anatomy without requiring a large number of prosthetic joint components. Modularly attached components involve additional joint interfaces between the components and improving the connection between components better stabilizes the joint assembly and improves the longevity of the joint. The present mounting boss improves the stability of a modularly attached component such as a sleeve or stem by reducing the risk of the mounting boss moving or loosening over time.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1 is a drawing which shows an artificial knee joint with a mounting boss used to attach an intramedullary component.

FIG. 2 is a perspective drawing of the mounting boss.

FIG. 3 is a cross-sectional drawing of the mounting boss.

FIG. 4 is a side view drawing of a femoral component with the mounting boss.

FIG. 5 is a cross-sectional drawing of the femoral component and mounting boss.

FIG. 6 is a cross-sectional drawing of the femoral component and mounting boss.

FIG. 7 is a perspective view drawing of the mounting boss.

FIG. 8 is a bottom view drawing of the mounting boss.

FIG. 9 is a cross-sectional drawing of the mounting boss and mounting rail.

FIG. 10 is a cross-sectional drawing of the mounting boss and mounting rail.

FIG. 11 is a side view drawing of the mounting boss.

FIG. 12 is a perspective drawing of the mounting boss.

FIG. 13 is a cross-sectional drawing of the mounting boss and mounting rail.

FIG. 14 is a cross-sectional drawing of the mounting boss and mounting rail.

FIG. 15 is a side view drawing of the mounting boss.

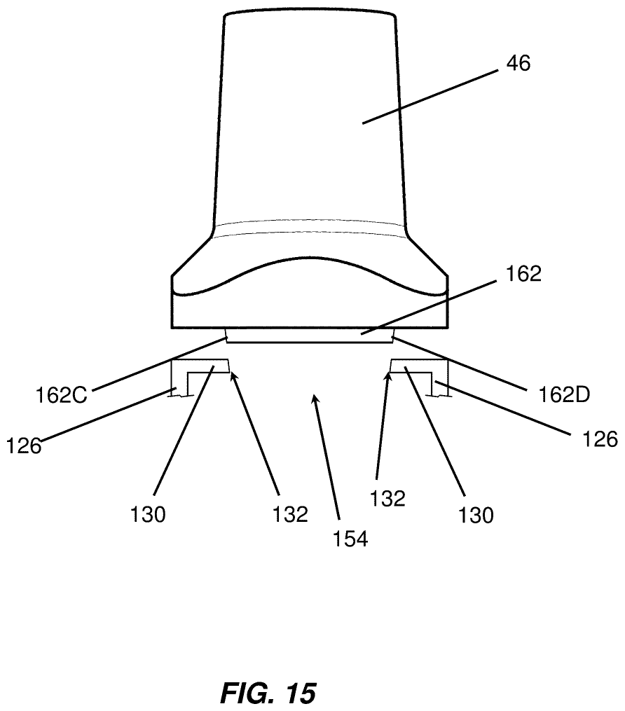

FIG. 16 is a side view drawing of the mounting boss.

Corresponding reference characters indicate corresponding components throughout the several views of the drawings. Unless otherwise noted, the drawings have been drawn to scale. Skilled artisans will appreciate that elements in the figures are illustrated for simplicity and clarity. For example, the dimensions of some of the elements in the figures may be exaggerated relative to other elements to help improve understanding of various examples of the present invention. Also, common but well-understood elements that are useful or necessary in a commercially feasible embodiment are often not depicted in order to facilitate a less obstructed view of these various embodiments of the present invention.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

In the above disclosure, reference has been made to the accompanying drawings, which form a part hereof, and in which are shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples. It is appreciated that the figures provided herewith are for explanation purposes to persons ordinarily skilled in the art.

As used herein, “adjacent” refers to near or close sufficient to achieve a desired effect. Although direct contact is common, adjacent can broadly allow for spaced apart features. As used herein, the singular forms “a,” and, “the” include plural referents unless the context clearly dictates otherwise.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be such as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a number or numerical range endpoint by providing that a given value may be a significant digit above or a significant digit below the number or endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range.

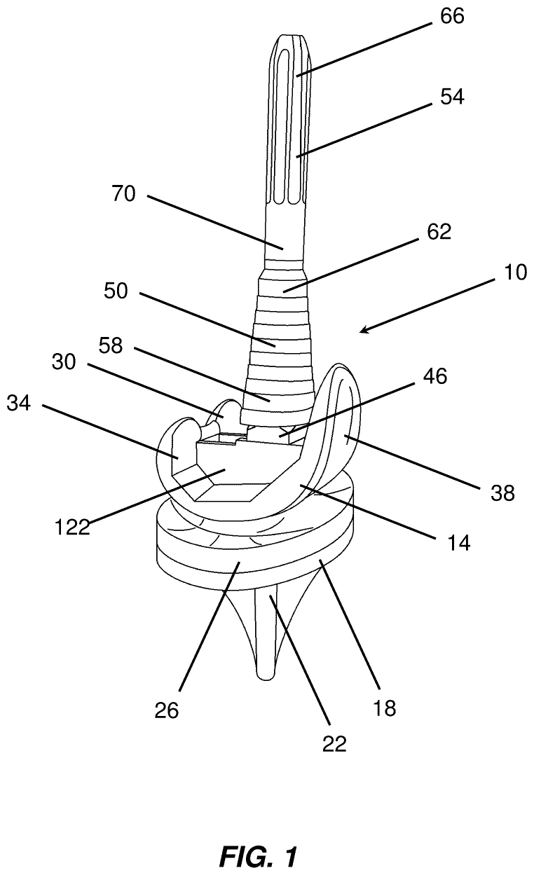

The disclosure particularly describes a mounting boss that is used to attach an intramedullary bone attachment component such as a bone attachment sleeve or stem to a prosthetic joint component. In discussing the mounting boss and prosthetic joint, terms such as proximally and distally are used to refer to the joint in an upright “installed” orientation as if the joint was installed into the knee joint of a patient with the patient standing in a vertical orientation. FIG. 1 shows example components of a prosthetic knee joint 10 including a femoral component 14 and a tibial component 18 which may include a tibial tray 22 with a lower bone attachment surface and a tibial bearing insert 26 with an upper bearing (articulation) surface. The femoral component 14 includes a medial condyle 30, a lateral condyle 34, and an anterior flange 38. When the prosthetic joint 10 is installed, the medial condyle 30 and lateral condyle 34 articulate against the tibial bearing insert 26 and the anterior flange 38 articulates against the patella to replicate the motion of the natural knee. The femoral component 14 includes an intercondylar box 122 that spans between the distal portions of the medial condyle 30 and lateral condyle 34. The intercondylar box 122 includes medial and lateral sidewalls that extend proximally from the condyles and an upper mounting surface with mounting rails that is used to attach a mounting boss 46 to the femoral component 14. The mounting boss 46 is in turn used to attach an intramedullary component such as a bone attachment sleeve 50 and/or stem 54 to the prosthetic knee joint component. The mounting boss 46 allows the surgeon to customize the angle and placement of the sleeve 50 and stem 54 to fit a particular patient. The modular assembly of the mounting boss 46, sleeve 50, and stem 54 allows the artificial joint to best fit the patient without requiring a large inventory or custom manufactured joint components.

The prosthetic knee joint 10 may be used in repairing a diseased or damaged knee joint. The tibial component 18 may be attached to a proximal end of a resected and prepared tibia and the femoral component 14 may be attached to a distal end of a resected and prepared femur. In some cases, a surgeon may desire to use a bone attachment sleeve 50 or a stem 54 in combination with a prosthetic joint component such as femoral component 14 to secure the prosthetic joint component to the leg bone. This may occur in cases with more bone damage or in revision surgeries where a failed prosthetic joint is removed and replaced. In these situations, a bone attachment sleeve 50 or stem 54 provides a larger mounting surface for attaching the prosthetic joint component within the bone canal.

The bone attachment sleeve 50 includes a generally conical body with a rough or porous outer surface that is suitable for bone ingrowth or cement fixation. The sleeve 50 often has an oval or rounded cross-sectional exterior shape and has a larger distal end 58 and a smaller proximal end 62. The sleeve provides a larger mounting surface for securing the prosthetic joint component 14 and is useful where the surgeon desires to broach the intramedullary canal and fix the joint component thereto. The stem 54 may include a proximal portion 66 and a distal end 70. The proximal portion 66 of the stem 54 is configured to be received in a cavity formed in the intramedullary canal at the distal end of the femur to provide stability to the prosthetic joint component 14. The stem 54 may be configured to be press-fitted or cemented within the cavity formed in the intramedullary canal. The proximal portion 66 of the stem body may also have flutes to inhibit movement of the stem 54 within the intramedullary canal. The distal end 70 of the stem 54 may include a shank with threads to allow the stem 54 to be removably attached to the sleeve 50 or the mounting boss 46.

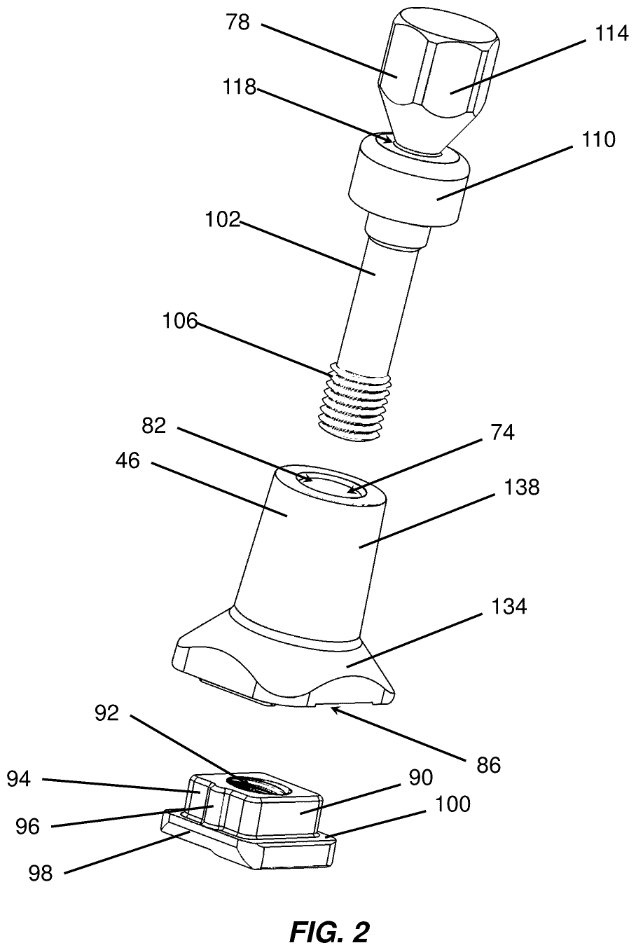

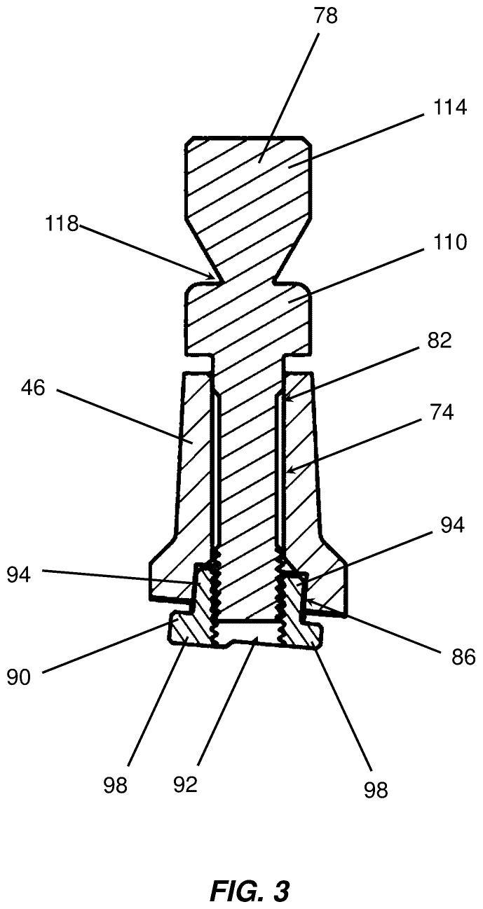

FIGS. 2 and 3 show a perspective view and a cross-sectional view of the mounting boss 46 and associated T-nut and bolt. FIG. 2 shows the disassembled components while FIG. 3 shows the mounting boss components in an assembled configuration. The body of the mounting boss 46 includes a through bore 74 which is used to attach the mounting boss 46 to the joint component. The through bore 74 includes a cylindrical upper bore section 82 which receives a bolt 78 and a square lower bore section 86 which receives the top of a T-nut 90. The bolt 78 passes through the boss body and threads into a threaded center hole 92 in the T-nut 90 to draw the T-nut towards the mounting boss body. The T-nut 90 includes a square upper post 94 that is a complementary shape and size to the lower bore section 86. The T-nut 90 also includes a square lower shoulder 98 that extends outwardly from the post 94 and forms a proximal clamping surface 100. The square upper post 94 engages the square lower bore section 86 and keeps the T-nut 90 from rotating while it is drawn upwardly into the boss 46 while tightening the bolt 78. The square upper post 94 may include a protrusion 96 that fits a complementarily shaped mating recess in the lower bore section 86 to index the T-nut 90 so that it only fits in a single orientation into the boss lower bore section 86.

The bolt 78 includers a shank 102 with a threaded distal end 106, a fastening head 110, and a drive head 114 which is separated from the fastening head 110 by a V-groove 118. The fastening head 110 is drawn against the proximal end of the mounting boss 46 and pushes the mounting boss 46 against the T-nut 90 and femoral component mounting rails. The V-groove 118 is sized to leave a reduced cross-section groove in the bolt such that the drive head 114 shears away from the fastening head 110 once a predetermined fastening torque has been reached. The drive head 114 is formed with an external or internal drive interface such as an external hex head or an internal hex or star socket to interface with a drive tool used to tighten the bolt 78. The fastening head 110 is made with a rounded outer peripheral side and shoulder that do not interface with a drive tool; preventing removal of the bolt 78 once the bolt has been properly torqued while attaching the mounting boss 46 to the joint component 14.

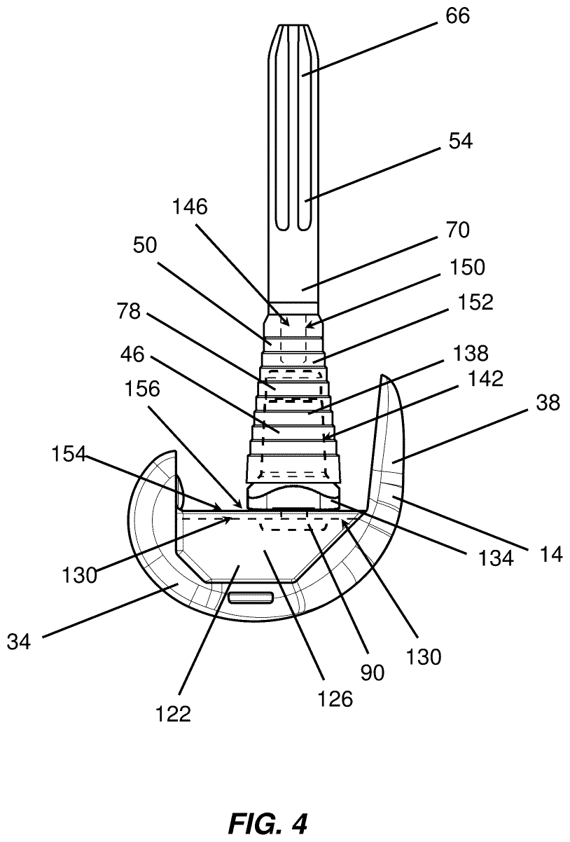

FIG. 4 shows a side view of the mounting boss 46 attached to a femoral component 14. The mounting boss 46 is used to attach a sleeve 50 and stem 54 to the femoral component 14. The femoral component 14 includes an intercondylar box 122 between the medial condyle 30 and lateral condyle 34. The intercondylar box 122 includes sidewalls 126 that are attached to the medial condyle 30 and lateral condyle 34 and that extend proximally from the condyles 30, 34. The example sidewalls 126 extend anteriorly to the anterior flange 38 and posteriorly to the posterior portions of the medial and lateral condyles 30, 34. The intercondylar box 122 includes mounting rails 130 attached to the proximal ends of the sidewalls 126. The mounting rails 130 extend inwardly from the sidewalls 126 towards the center of the femoral component 14 and intercondylar box 122 to create mounting rails that overhang the sidewalls 126. The medial and lateral mounting rails 130 define a central mounting slot 154 therebetween which is open adjacent the posterior end of the femoral component 14. The medial and lateral mounting rails 130 are joined to each other and to the anterior flange 38 at the anterior end of the femoral component 14. The mounting rails 130 form the flat proximal surface 156 of the intercondylar box 122 and define a planar surface to which the mounting boss 46 is attached.

The mounting boss 46 is attached to the mounting rails 130. The mounting boss 46 includes a generally square or rectangular distal end 134 which is attached to the proximal surface defined by the mounting rails 130. The proximal exterior 138 of the mounting boss 46 has a tapered conical surface 138, such as morse taper surface. The bolt 78 passes through the mounting boss bore 74 and threads into the T-nut 90. The T-nut 90 is positioned with its proximal square upper post 94 passing through the slot 154 between mounting rails 130 and into the square lower bore section 86 of the mounting boss. The T-nut distal lower shoulder 98 is positioned distally beneath the mounting rails 130. The T-nut is drawn against the mounting rails 130 by the bolt 78. The mounting rails 130 are held between the T-nut lower shoulder 98 and the mounting boss distal end 134.

The bone attachment sleeve 50 includes a tapered inner surface 142 with a mating taper that matches the taper 138 formed on the mounting boss 46. The bone attachment sleeve 50 is attached to the mounting boss 46 after attaching the mounting boss 46 to the femoral component 14 by sliding its tapered inner bore 142 over the mounting boss 46 and pressing the tapered inner surface 142 against the tapered outer surface 138 of the mounting boss 46 with sufficient force to join the sleeve 50 to the mounting boss 46. The stem 54 includes a distal mounting post 146 which is attached to a corresponding mounting socket 150 in the sleeve 50. The stem mounting post 146 may be threaded and the socket 150 may be threaded. Alternatively, the stem mounting post 146 may include a taper and the mounting socket 150 may include the corresponding tapered bore. If the stem 54 includes a threaded mounting post 146, the stem 54 may be used to attach the mounting boss 46 to the femoral component 14 instead of the bolt 78 if a sleeve 50 is not used.

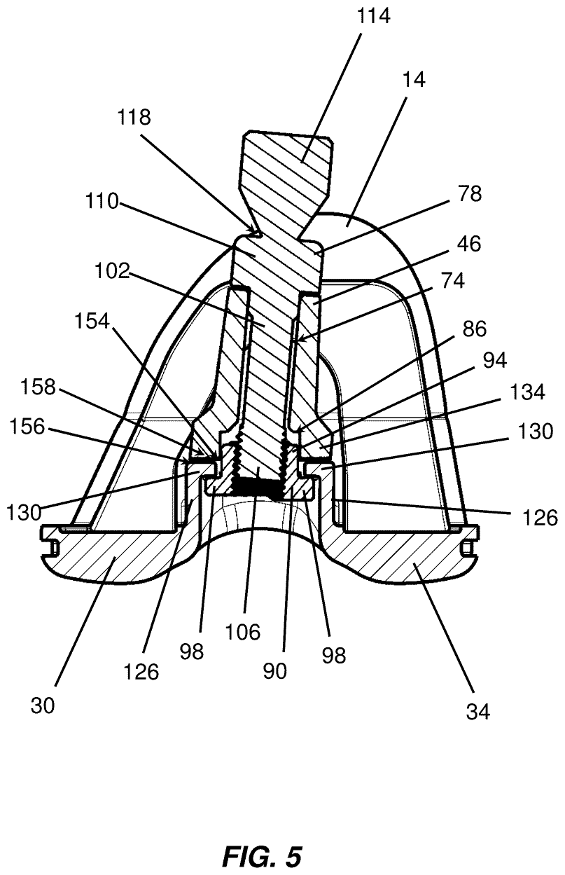

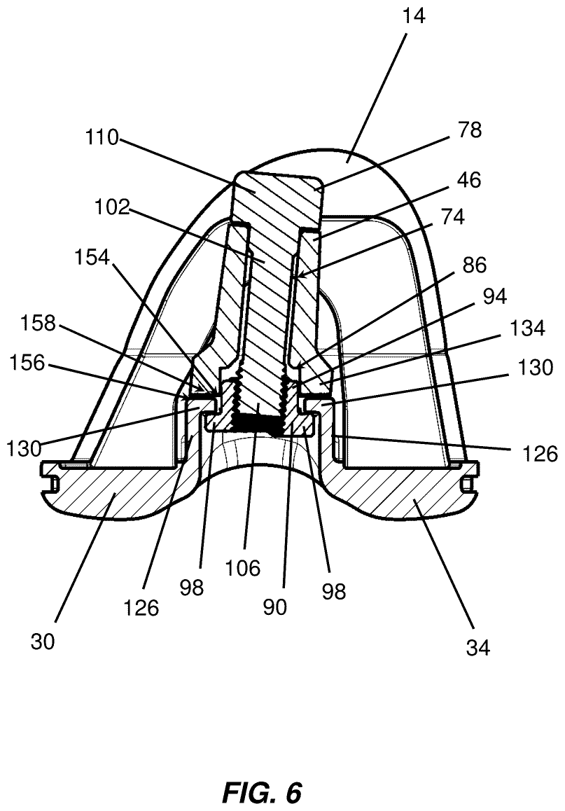

FIGS. 5 and 6 show cross-sectional drawings taken through the femoral component 14 and the mounting boss 46. The distal square section 134 of the mounting boss 46 is seated on the proximal surface of the mounting rails 130. The square upper post 94 of the T-nut 90 extends through the mounting slot 154 is inserted into the square lower bore section 86 of the mounting boss 46. The T-nut 90 square lower shoulder 98 is positioned distally beneath the mounting rails 130 and the mounting rails 130 are held between the distal end 134 of the mounting boss 46 and the T-nut lower shoulder 98. The bolt shank 102 passes through the mounting boss bore 74 and the threaded end 106 engages the threads in the T-nut 90. The T-nut 90 has been drawn upwardly into the mounting boss 46 by the bolt 78. The slot 154 between the mounting rails is slightly wider than the upper post 94 of the T-nut 90 so that the T-nut upper post 94 fits in the slot. The T-nut lower flange/shoulder 98 is wider than the slot 154 and engages the distal surfaces of the mounting rails 130. As the bolt 78 is tightened, the mounting rails 130 are clamped between the T-nut 90 and the mounting boss 46.

As shown, the mounting boss 46 may be manufactured with an angled distal end 134 so that it is oriented at an angle relative to the femoral component intercondylar box 122 when attached to the femoral component 14 and mounts the sleeve 50 and stem 54 at an angle relative to the femoral component 14.

FIG. 5 shows a configuration where the bolt 78 has not been fully tightened. FIG. 6 shows a configuration where the bolt 78 has been fully tightened and the drive head 114 has sheared from the fastener head 110 at the location of groove 118. The fastener head 110 is held against the proximal end of the mounting boss 46 and the bolt 78 holds the distal end 134 of the mounting boss 46 against the proximal surface of the mounting rails 130 and holds the T-nut 90 against the distal surface of the mounting rails 130. The sleeve 50 may be constructed such that the sleeve is not mountable to the mounting boss 46 until the drive head 114 has been removed from the bolt 78. For example, the sleeve 50 may have an inwardly extending projection such as inwardly extending shoulder 152 near the top of the interior tapered bore 142 with sufficient interior space for the fastening head 110 and with insufficient interior space to accommodate the drive head 114 while seating the sleeve 50 on the mounting boss 46. If the sleeve 50 was installed onto the mounting boss 46 and bolt 78 before removal of the drive head 114 from the bolt 78, the projection 152 would contact the drive head 114 and prevent the sleeve 50 from attaching to the mounting boss 46. This inwardly extending projection structure requires that the bolt 78 is fully torqued and the mounting boss 46 is properly secured to the femoral component 14 before the sleeve 50 is attached to the mounting boss 46.

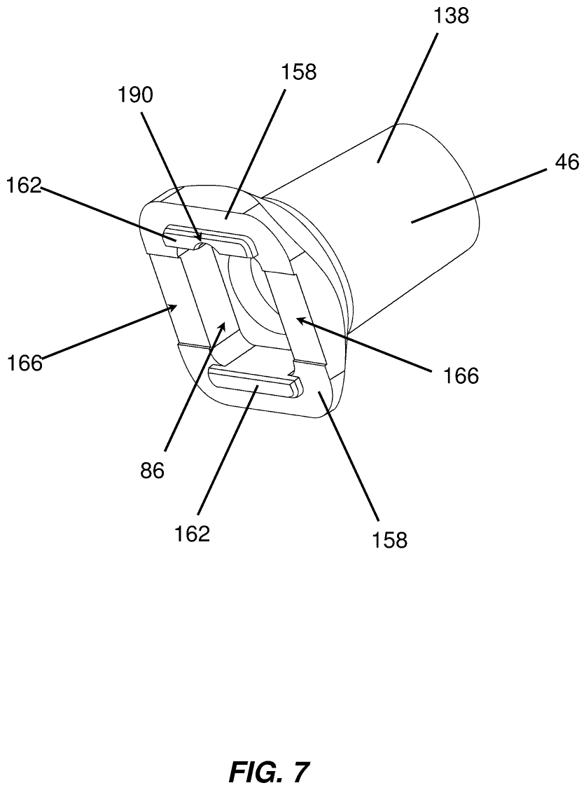

FIG. 7 shows a bottom perspective view of the mounting boss 46. The distal end 134 of the mounting boss 46 includes a flat distal mounting surface 158 which is seated on the flat proximal face 156 of the mounting rails 130 during use. The mounting boss distal mounting surface 158 is modified with mounting features that deform the femoral component mounting rails 130 when the mounting boss 46 is attached to the femoral component 14. The mounting features include anterior and posterior anti-rotation tabs 162 which extend distally beyond the mounting surface 158. When the mounting boss 46 is installed onto the joint component 14, the anti-rotation tabs 162 extend between the mounting rails 130, extending across the slot 154 between the mounting rails. The anti-rotation tabs 162 each have an overall length between the opposed ends of the tabs that engage the mounting rails 130 which is greater than the width of the slot 154 between the mounting rails 130. Accordingly, the mounting rails 130 are bent/deformed by the anti-rotation tabs 162 when the mounting boss 46 is installed on the femoral joint component 14. As the mounting boss 46 is drawn into contact with the femoral component 14, the anti-rotation tabs 162 are pressed into the slot 154 between the mounting rails 130 and deform the inwardly facing edges of the mounting rails 130 outwardly around the anti-rotation tabs 162. This deformation of the mounting rails results in residual pressure between the mounting rails 130 and anti-rotation tabs 162 and inhibits relative motion between the mounting boss 46 and the femoral component 14.

The mounting boss 46 also includes medial and lateral recesses 166 located along the medial and lateral sides of the distal mounting surface 158. The medial and lateral recesses 166 extend proximally into the distal mounting surface 158 and define recesses in the mounting face that contacts the mounting rails 130. When the mounting boss 46 is installed onto the joint component 14, the medial and lateral recesses 166 are positioned along the mounting rails 130. As the mounting rails 130 are clamped between the distal mounting surface 158 and the T-nut lower shoulder 98, adjacent sections of the medial and lateral mounting rails 130 are bent/deformed proximally into the medial and lateral recesses 166 as the adjacent sections of the mounting rails are forced proximally by the T-nut lower shoulder 98. The medial and lateral recesses 166 are formed with a sharp corner where their anterior and posterior ends meet the distal mounting surface 158 and the mounting rails 130 are bent around this corner. The deformation of the mounting rails 130 around the medial and lateral recesses 166 and the resulting residual pressure between these structures inhibits relative motion between the mounting boss 46 and the femoral component 14.

In attaching the mounting boss 46 to the femoral component 14, a mounting boss with a desired medial or lateral angle may be chosen to fit the patient anatomy. The mounting boss 46 may be moved anteriorly or posteriorly within the slot 154 between mounting rails 130 to a position that best fits the patient anatomy. Once a desired configuration and location is achieved, the bolt 78 is tightened until the drive head 114 shears from the fastening head 110 and the mounting boss 46 is permanently attached to the femoral component. The mounting rails 130 are deformed by the anti-rotation tabs 162 and the recesses 166 as the mounting rails 130 are clamped and held between the distal mounting surface 158 of the mounting boss 46 and the shoulder 98 of the T-nut 90. Because of the permanent deformation of the mounting rails 130 when the mounting boss 46 is properly attached to the joint component 14, the mounting boss 46 should not moved to another location along the femoral component 14. The deformation of the mounting rails 130 may reduce the stability of the joint between the mounting boss 46 and the mounting rails 130 if the mounting boss 46 is moved and reattached in a different location along the mounting rails 130. The break-away drive head 114 is thus advantageous in providing a safety measure that prevents a surgeon from removing and reattaching the mounting boss 46 once the bolt 78 is properly torqued to attach the mounting boss 46 to the joint component 14. Use of a sleeve 50 with an inwardly extending projection 152 that prevents attachment of the sleeve 50 to the mounting boss before removal of the drive head 114 further ensures that a user properly attaches the mounting boss 46 to the joint component 14 before attachment of the sleeve 50 to the mounting boss 46.

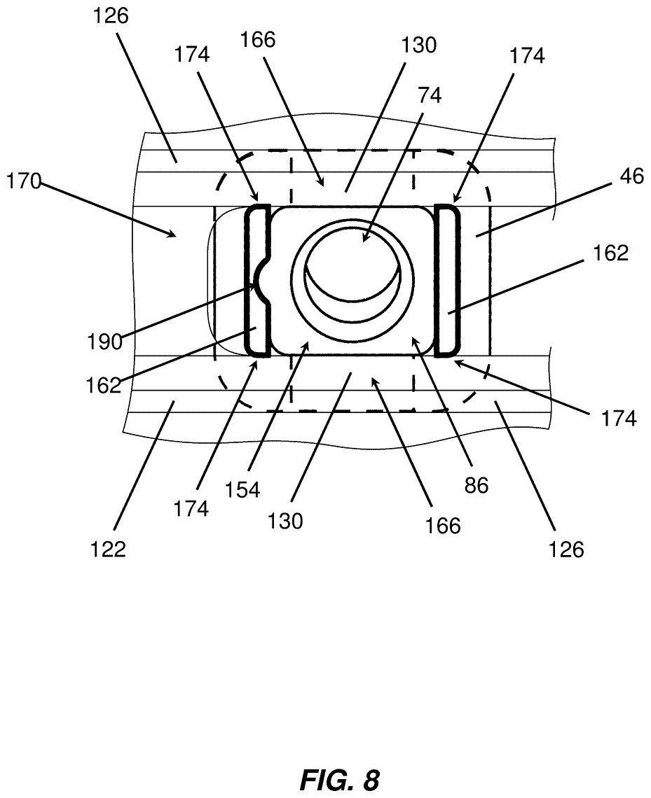

FIG. 8 shows a bottom view of the joint component intercondylar box 122 with the mounting boss 46 attached to the proximal mounting surface 156 of the intercondylar box 122. The medial and lateral box sidewalls 126 are visible with the mounting rails 130 extending inwardly from the sidewalls 126. The mounting rails 130 are joined together at the anterior end of the box 122 as indicated at area 170. The mounting rails 130 define a slot 154 at the posterior end of the box 122. The mounting boss 46 is attached to the upper (proximal) mounting surface defined by the mounting rails 130 with the distal mounting surface 158 of the mounting boss 46 against the mounting rails 130. The anti-rotation tabs 162 are inserted between the mounting rails 130 in the slot 154 and are held in this position by the T-nut 90 and bolt 78 (both not shown for clarity). The anti-rotation tabs 162 deform the inwardly facing sides of the mounting rails 130 at locations 174. This deformation results in residual contact stress between the anti-rotation tabs 162 and the mounting rails 130 and inhibits movement of the attached mounting boss 46. FIG. 8 also shows the medial and lateral recesses 166 and shows how these overlap a section of the mounting rails 130 located between the anti-rotation tabs 162.

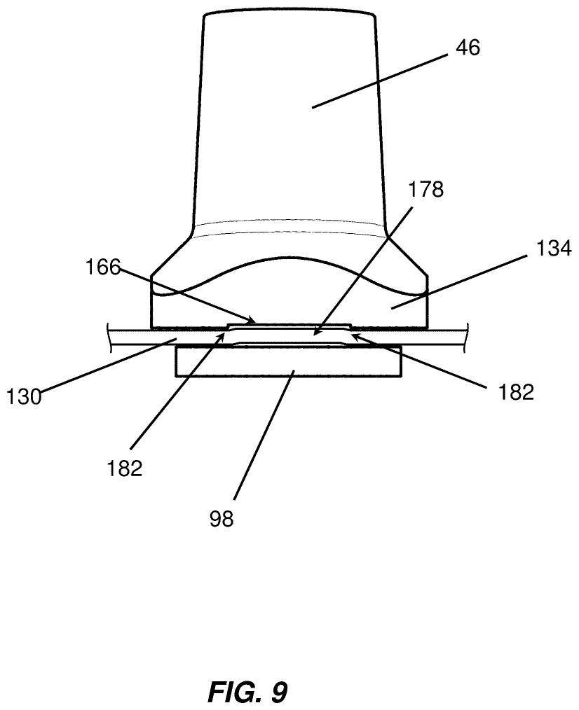

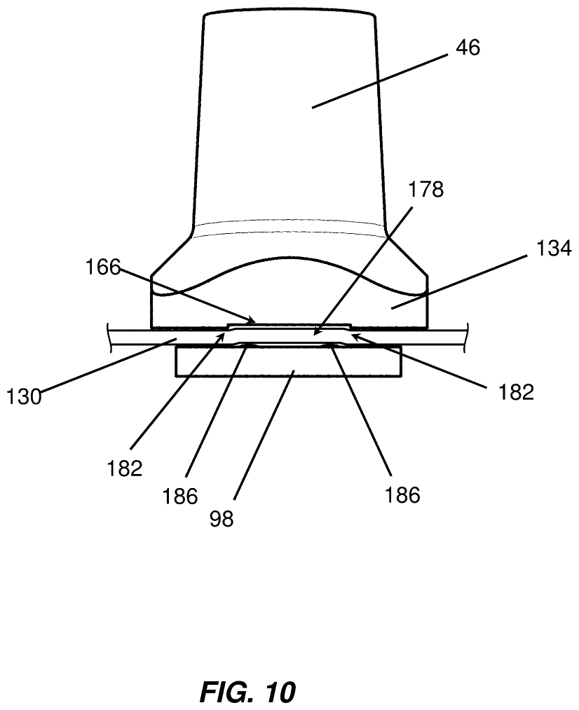

FIG. 9 shows a side cross-sectional view of the mounting boss 46, mounting rails 130, and T-nut 90. The view shows a cross-section through one of the medial or lateral recesses 166. This drawing illustrates how the mounting rail 130 bends upwardly into the recess 166 when it is clamped between the mounting boss 46 and the T-nut 90. The pressure applied to the mounting rail 130 by the T-nut lower shoulder 98 causes the mounting rail 130 to bend/deform upwardly (proximally) into the recess 166 as indicated at area 178. If the joint between the mounting boss 46 and the joint component 14 is disassembled, the mounting rail 130 will typically exhibit a crease in the mounting rail 130 adjacent the ends of each recess at the locations indicated by 182. FIG. 10 shows a drawing which is similar to FIG. 9 except that the T-nut 90 has small protrusions 186 formed on the medial and lateral sides of the proximal clamping face 100 of the lower shoulder 98. The protrusions 186 aid in bending the mounting rails 130 into the recesses 166.

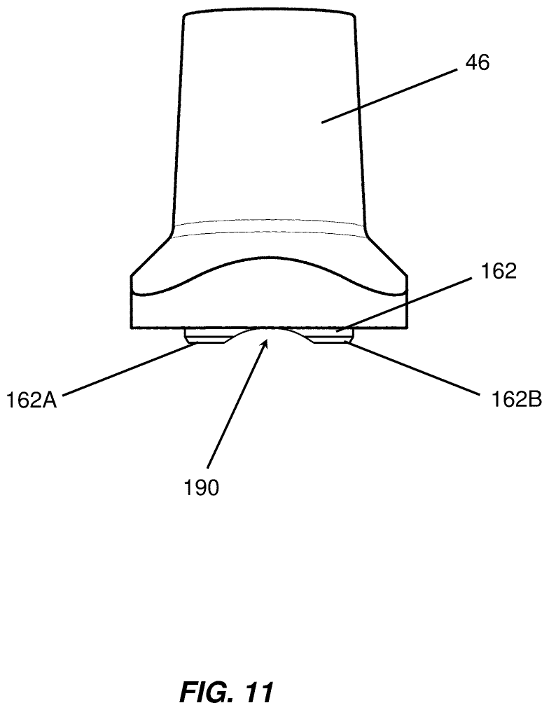

FIG. 11 shows a front view drawing of the mounting boss 46. This figure illustrates how the anti-rotation tabs 162 may have a shape that does not extend continuously across the mounting boss 46. The anti-rotation tabs 162 may include a recess 190 or other interruption to the shape of the tab 162. The recess 190 separates the anti-rotation tab 162 into a first anti-rotation tab section 162A that contacts the medial mounting rail 130 and a second anti-rotation tab section 162B that contact the lateral mounting rail 130 as discussed above.

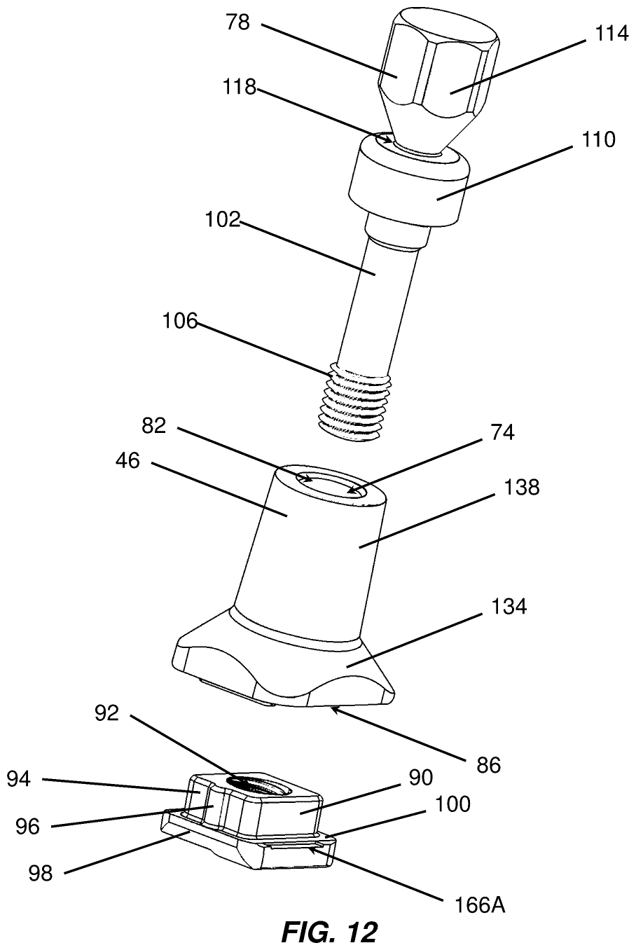

FIG. 12 shows another perspective view of the mounting boss 46 and associated T-nut and bolt. FIG. 12 shows the disassembled components. The body of the mounting boss 46 includes a through bore 74 which is used to attach the mounting boss 46 to the joint component. The through bore 74 includes a cylindrical upper bore section 82 which receives a bolt 78 and a square lower bore section 86 which receives the top of a T-nut 90. The bolt 78 passes through the boss body and threads into a threaded center hole 92 in the T-nut 90 to draw the T-nut towards the mounting boss body. The T-nut 90 includes a square upper post 94 that is a complementary shape and size to the lower bore section 86. The T-nut 90 also includes a square lower shoulder 98 that extends outwardly from the post 94. The square upper post 94 engages the square lower bore section 86 and keeps the T-nut 90 from rotating while it is drawn upwardly into the boss 46 while tightening the bolt 78. The square upper post 94 may include a protrusion 96 that fits a complementarily shaped mating recess in the lower bore section 86 to index the T-nut 90 so that it only fits in a single orientation into the boss lower bore section 86.

The mounting boss 46 and T-nut 90 differ from those discussed above in that the recess 166 is formed in the T-nut 90 instead of the base. The proximal clamping surface 100 of the T-nut lower shoulder 98 includes medial and lateral recesses 166A. The medial and lateral recesses 166A are located along the proximal faces of the medial and lateral sides of the lower shoulder 98. The medial and lateral recesses 166A extend distally into the T-nut shoulder 98 and define recesses in the clamping face that contacts the mounting rails 130. When the mounting boss 46 and T-nut 90 are installed onto the joint component 14, the medial and lateral recesses 166A are positioned along the mounting rails 130. As the mounting rails 130 are clamped between the distal mounting surface 158 of the mounting boss 46 and the T-nut lower shoulder 98, adjacent sections of the medial and lateral mounting rails 130 are bent/deformed distally into the medial and lateral recesses 166A as the adjacent sections of the mounting rails are forced distally by the mounting boss distal mounting surface 158. The medial and lateral recesses 166A are formed with a sharp corner where their anterior and posterior ends meet the proximal clamping surface 100 and the mounting rails 130 are bent around this corner. The deformation of the mounting rails 130 around the medial and lateral recesses 166A and the resulting residual pressure between these structures inhibits relative motion between the mounting boss 46 and the femoral component 14.

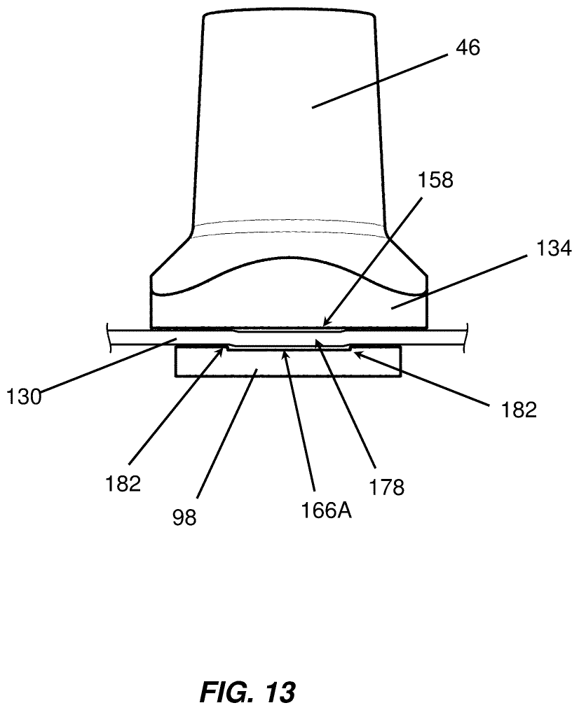

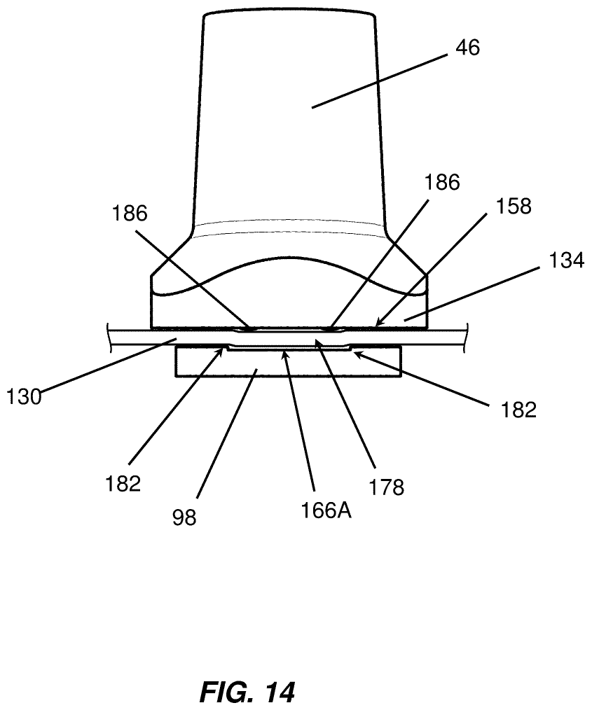

FIG. 13 shows a side cross-sectional view of the mounting boss 46, mounting rails 130, and T-nut 90. The view shows a cross-section of the mounting rail 130 as it extends over one of the medial or lateral recesses 166A. This drawing illustrates how the mounting rail 130 bends downwardly into the recess 166A when it is clamped between the mounting boss 46 and the T-nut 90. The pressure applied to the mounting rail 130 by the mounting boss distal mounting surface 158 causes the mounting rail 130 to bend/deform downwardly (distally) into the recess 166A as indicated at area 178. If the joint between the mounting boss 46 and the joint component 14 is disassembled, the mounting rail 130 will typically exhibit a crease in the mounting rail 130 adjacent the ends of each recess at the locations indicated by 182. FIG. 14 shows a drawing which is similar to FIG. 13 except that the mounting boss 46 has small protrusions 186 formed on the medial and lateral sides of the distal mounting surface 158. The protrusions 186 aid in bending the mounting rails 130 into the recesses 166A.

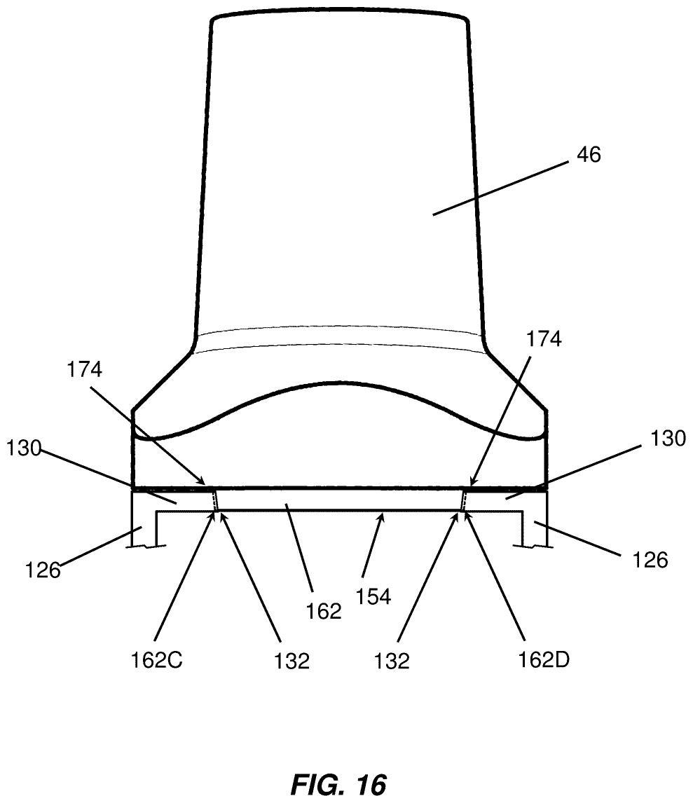

FIG. 14 shows a front view drawing of the mounting boss 46 and a portion of the joint component 14 including the mounting rails 130 and the medial and lateral box sidewalls 126. This figure illustrates how the anti-rotation tabs 162 may have inwardly beveled ends 162C, 162D that contact the mounting rails 130. As discussed, the distal end 134 of the mounting boss 46 includes a flat distal mounting surface 158 which is seated on the flat proximal face 156 of the mounting rails 130 during use. The inwardly facing sides of the mounting rails 130 typically include flat and vertically oriented faces, but may also include rounded or chamfered edges and may also include inwardly sloping inner sides as illustrated in FIG. 14. The sloped ends 162C, 162D of the anterior and posterior anti-rotation tabs 162 engage the inward sides of the mounting rails 130. When the mounting boss 46 is installed onto the joint component 14 as shown in FIG. 16, the anti-rotation tabs 162 extend between the mounting rails 130, extending across the slot 154 between the mounting rails. The anti-rotation tabs 162 each have an overall length between the opposed ends 162C, 162D of the tabs that engage the mounting rails 130 which is greater than the width of the slot 154 between the mounting rails 130. Accordingly, the mounting rails 130 are bent/deformed by the ends of the anti-rotation tabs 162 when the mounting boss 46 is installed on the femoral joint component 14. As the mounting boss 46 is drawn into contact with the femoral component 14, the anti-rotation tabs 162 are pressed into the slot 154 between the mounting rails 130 and press into the inwardly facing edges 132 of the mounting rails 130 and deform the inwardly facing edges 132 of the mounting rails 130 outwardly around the anti-rotation tabs 162. This deformation of the mounting rails results in residual pressure between the mounting rails 130 and anti-rotation tabs 162 and inhibits relative motion between the mounting boss 46 and the femoral component 14.

The mounting boss 46 or T-nut 90 also includes medial and lateral recesses 166, 166A located along the medial and lateral sides of the distal mounting surface 158 or proximal clamping surface 100 (extending perpendicular to the plane of the drawing on the medial and lateral sides). The medial and lateral recesses 166 extend proximally into the distal mounting surface 158 or distally into the proximal clamping surface 100 and define recesses in the face that contacts the mounting rails 130. When the mounting boss 46 is installed onto the joint component 14, the medial and lateral recesses 166, 166A are positioned along the mounting rails 130. As the mounting rails 130 are clamped between the distal mounting surface 158 and the T-nut lower shoulder 98 proximal clamping face 100, adjacent sections of the medial and lateral mounting rails 130 are bent/deformed proximally or distally into the medial and lateral recesses 166, 166A as the adjacent sections of the mounting rails are forced proximally by the T-nut lower shoulder 98. The medial and lateral recesses 166, 166A are formed with a sharp corner where their anterior and posterior ends meet the distal mounting surface 158 or proximal clamping surface 100 and the mounting rails 130 are bent around this corner. The deformation of the mounting rails 130 around the medial and lateral recesses 166 and the resulting residual pressure between these structures inhibits relative motion between the mounting boss 46 and the femoral component 14.

In attaching the mounting boss 46 to the femoral component 14, a mounting boss 46 with a desired medial or lateral angle may be chosen to fit the patient anatomy. The mounting boss 46 may be moved anteriorly or posteriorly within the slot 154 between mounting rails 130 to a position that best fits the patient anatomy. Once a desired configuration and location is achieved, the bolt 78 is tightened until the drive head 114 shears from the fastening head 110 and the mounting boss 46 is permanently attached to the femoral component. The mounting rails 130 are deformed by the anti-rotation tabs 162 and/or the recesses 166A as the mounting rails 130 are clamped and held between the distal mounting surface 158 of the mounting boss 46 and the proximal clamping surface 100 of the shoulder 98 of the T-nut 90. Anti-rotation tabs 162 are drawn between the mounting rails 130 in the slot 154 and are held in this position by the T-nut 90 and bolt 78 (both not shown for clarity). The anti-rotation tabs 162 deform the inwardly facing sides of the mounting rails 130 at locations 174. This deformation results in residual contact stress between the anti-rotation tabs 162 and the mounting rails 130 and inhibits movement of the attached mounting boss 46. The mounting rails 130 are deformed into the recesses 166, 166A.

As is described above, the mounting boss 46 is attached to the joint component at a boss attachment joint. The mounting boss 46 and T-nut 90 may together create the attachment joint. The attachment joint engages the mounting rails 130 and includes a mounting feature that deforms the mounting rails 130. The attachment interface includes one or more mounting features such as anti-rotation tabs 162 and recesses 166, 166A that deform the mounting rails 130 when the mounting boss 46 is attached to a joint component 14. In many examples, the attachment interface includes anti-rotation tabs 162 that extend between the mounting rails 130 and recesses that are positioned against the face of the mounting rails 130. The mounting rails 130 are deformed around the anti-rotation tabs 162 and the mounting rails 130 are deformed into the recesses 166, 166A. Deformation of the mounting rails 130 at the mounting features keys the mounting features into the mounting rails 130 and creates residual pressure between the mounting rails 130 and mounting features and a more secure attachment interface between the mounting boss 46 and the joint component 14.

The inventors have determined that the stresses placed on the interface between prior art femoral stem adjustable mounting bosses and the femoral component may cause the mounting boss to move relative to the femoral component. The back and forth bending forces placed on the stem can cause the mounting boss to pivot and walk from the small axial rotational slipping between the mounting boss and joint component and the shear forces placed on the stem and mounting box. Forces placed on a stem or sleeve may cause the mounting boss to place more contact force between one mounting boss corner and a mounting surface on the joint component and a reduced force between the other corners and the joint component which may allow the other corners to shift relative to the joint component. This can cause movement and loosening of the mounting boss relative to the femoral component.

The present mounting boss 46 significantly reduces or eliminates movement or loosening of the mounting boss 46 relative to the joint component 14. The anti-rotation tabs 162 and recesses 166 create deformation and mating edges which key the mounting boss 46 into the joint component mounting rails 130 after assembly and also provide locations of increased contact pressure and increased friction between the mounting boss 46 and the joint component 14. This interface significantly reduces movement and loosening of the mounting boss 46 from the joint component 14.

In use, the femur may be surgically prepared for receiving the prosthetic femoral component 14 in a manner known in the art. The prosthetic joint 10 may be used in primary or revision knee replacement procedures. A bone attachment sleeve 50 or stem 54 may be selected as needed with a suitable size for the patient, and a mounting boss 46 may be selected to provide the desired angle of attachment of the sleeve 50 and stem 54 relative to the joint component 14. The mounting boss 46 may be placed between the condyles 30, 34 near the posterior side of the joint component at the opening of the slot 154 with the mounting boss 46 on the proximal side of the mounting rails 130 and with the T-nut 90 on the distal side of the mounting rails 130. The mounting boss 46 may then be moved toward the anterior side of the joint component 14 to the desired position along the mounting rails 130. The bolt 78 is then tightened to clamp the mounting rails 130 between the mounting boss 46 and the T-nut 90; deforming the mounting rails 130 as discussed above. If a break away bolt 78 is used, the drive head 114 will shear at groove 118 when the fastening torque is reached. Once the mounting boss 46 is attached to the joint component 14, a sleeve 50 may be attached to the mounting boss 46 by positioning the sleeve inner tapered surface over the mounting boss outer taper 138 and applying sufficient force to properly attach the sleeve 50. A stem 54 can then be fastened to the proximal end of the sleeve 50. In some embodiments, a stem 54 with a threaded distal shaft may be used in place of a bolt 78 to attach the mounting boss 46 to the femoral joint component 14. In this embodiment, the stem 54 may include a distal end that matches the shank 102 and threaded distal end 106 of the bolt 78. The mounting boss 46 and the associated sleeve 50 or stem 54 may be fixed to the femoral component 12 in an infinite number of positions along the anterior to posterior direction of the slot 38. The mounting boss 46 allows flexibility in positioning and attaching an intramedullary attachment member such as a sleeve 50 or stem 54 at a desired position and angle on a joint component while more securely attaching the intramedullary member and preventing later movement or loosening.

The above description of illustrated examples of the present invention, including what is described in the Abstract, is not intended to be exhaustive or to be limiting to the precise forms disclosed. While specific examples of the invention are described herein for illustrative purposes, various equivalent modifications are possible without departing from the broader scope of the present claims. Indeed, it is appreciated that specific example dimensions, materials, etc., are provided for explanation purposes and that other values may also be employed in other examples in accordance with the teachings of the present invention.

What is claimed is:

1. A mounting system for mounting an intramedullary bone attachment component to an artificial joint component comprising:
an artificial femoral joint component having a bone attachment surface and an articulation surface comprising a medial condyle and a lateral condyle;
an intercondylar box comprising a medial side wall attached to the medial condyle, a lateral side wall attached to the lateral condyle, a medial mounting rail attached to a proximal side of the medial side wall, and a lateral mounting rail which is attached to a proximal side of the lateral side wall, wherein the medial mounting rail and the lateral mounting rail extend towards a center of the intercondylar box and define a proximal mounting surface;
a mounting boss that is attachable to the medial mounting rail and the lateral mounting rail, wherein the mounting boss comprises a first distal mounting surface which is attachable to the proximal mounting surface of the femoral joint component, and wherein the mounting boss comprises a second mounting surface which is attachable to an intramedullary component to thereby attach the intramedullary component to the femoral joint component;
wherein the mounting boss first distal mounting surface comprises a mounting feature that deforms the medial and lateral mounting rails when the mounting boss is attached to the medial and lateral mounting rails to thereby inhibit movement of the mounting boss along the medial and lateral mounting rails after attachment of the mounting boss to the medial and lateral mounting rails.

2. The mounting system of claim 1, wherein the mounting feature causes permanent deformation of the medial and lateral mounting rails.

3. The mounting system of claim 2, wherein the mounting boss is attached to the femoral joint component with a bolt comprising a threaded shank, a fastening head that does not engage a drive tool, a drive head formed with a drive surface that engages a drive tool, and wherein the drive head separates from the fastening head when a predetermined fastening torque is reached.

4. The mounting system of claim 1, wherein the first distal mounting surface comprises a distal end of the mounting boss, wherein the distal end of the mounting box is attached to a proximal face of the medial and lateral mounting rails, wherein the device further comprises a T-nut, wherein the T-nut is located on a distal side of the medial and lateral mounting rails, and wherein the medial and lateral mounting rails are clamped between the T-nut and the distal end of the mounting boss.

5. The mounting system of claim 4, wherein the proximal mounting surface comprises a horizontal and planar surface.

6. The mounting system of claim 1, wherein the femoral joint component comprises a mounting slot defined between the medial mounting rail and the lateral mounting rail, wherein the mounting feature comprises an anti-rotation tab that extends between the medial mounting rail and the lateral mounting rail and wherein a length between ends of the anti-rotation tab is greater than a distance between the medial mounting rail and the lateral mounting rail such that the medial and lateral mounting rails are deformed when the anti-rotation tab is inserted between the medial and lateral mounting rails.

7. The mounting system of claim 1, wherein the mounting feature comprises a recess on the distal end of the mounting boss, wherein a mounting rail selected from the medial mounting rail and the lateral mounting rail extends across the distal end of the mounting boss and across the recess, and wherein the mounting rail is deformed into the recess when the mounting rail is clamped between the distal end of the mounting boss and the T-nut.

8. The mounting system of claim 7, wherein the T-nut comprises a protrusion located adjacent the recess and wherein the protrusion deforms the mounting rail into the recess.

9. The mounting system of claim 1, wherein the second mounting surface comprises a male tapered section that receives a female tapered section of an intramedullary bone attachment sleeve to thereby attach the bone ingrowth sleeve to the mounting boss, wherein the mounting boss is attached to the femoral joint component with a bolt comprising a threaded shank, a fastening head that does not engage a drive tool and that engages the mounting boss to secure the mounting boss to the femoral joint component, and a drive head formed with a drive surface that engages a drive tool, wherein the drive head separates from the fastening head when a predetermined fastening torque is applied to the drive head, and wherein the bone attachment sleeve comprises a projection located adjacent the fastening head which contacts the drive head and prevents installation of the sleeve if the drive head is not removed from the bolt.

10. The mounting system of claim 1, wherein the mounting boss is attachable to the proximal mounting surface at a variable anterior posterior position along the medial and lateral mounting rails.

11. A mounting system for mounting an intramedullary bone attachment component to an artificial joint component comprising:
an artificial joint component having a first side with a bone attachment surface and a second side with an articulation surface;
a first mounting rail attached to the first side of the artificial joint component, wherein the first mounting rail defines a joint component mounting surface;
a mounting boss that is attachable to the first mounting rail, wherein the mounting boss comprises a first mounting surface which is attachable to the joint component mounting surface, and wherein the mounting boss comprises a second mounting surface which is attachable to an intramedullary bone attachment component to thereby attach the intramedullary bone attachment component to the artificial joint component;
wherein the mounting boss first mounting surface is attached to the first mounting rail at a boss attachment joint that comprises a mounting feature that deforms the first mounting rail when the mounting boss is attached to the first mounting rail to thereby inhibit movement of the mounting boss relative to the first mounting rail.

12. The mounting system of claim 11, wherein the mounting feature causes permanent deformation of the first mounting rail.

13. The mounting system of claim 11, wherein the joint component comprises a second mounting rail which is attached to the first side of the artificial joint component, and wherein the mounting boss is attached to the second mounting rail at the boss attachment joint.

14. The mounting system of claim 11, wherein the device further comprises a T-nut, wherein the mounting boss is attached to a first side of the first mounting rail, wherein the T-nut is attached to a second side of the first mounting rail, wherein the first mounting rail is clamped between the T-nut and the mounting boss, wherein at least one of the mounting boss and the T-nut comprises a recess, wherein the first mounting rail extends across the recess, and wherein the first mounting rail is permanently deformed into the recess by the T-nut and mounting boss.

15. The mounting system of claim 11, wherein the device further comprises a T-nut, wherein the mounting boss is attached to a first side of the first mounting rail, wherein the T-nut is attached to a second side of the first mounting rail, wherein the first mounting rail is clamped between the T-nut and the mounting boss, wherein the T-nut is attached to the mounting boss with a bolt comprising a threaded shank, a fastening head that does not engage a drive tool, and a drive head formed with a drive surface that engages a drive tool, and wherein the drive head separates from the fastening head when a predetermined fastening torque is reached to inhibit removal of the bolt.

16. The mounting system of claim 13, wherein the joint component comprises a mounting slot defined between the first mounting rail and a second mounting rail, wherein the mounting feature comprises an anti-rotation tab that extends between the first mounting rail and the second mounting rail and wherein a length of the anti-rotation tab is greater than a distance between the first mounting rail and the second mounting rail such that the first and second mounting rails are deformed when the anti-rotation tab is inserted between the first and second mounting rails.

17. The mounting system of claim 11, wherein the mounting feature comprises a recess on the mounting boss first mounting surface, wherein the first mounting rail extends across the first mounting surface and across the recess, and wherein the mounting rail is deformed into the recess when the mounting rail is clamped against the mounting boss.

18. The mounting system of claim 17, wherein the first mounting rail is clamped between a T-nut and the mounting boss first mounting surface, and wherein the T-nut comprises a protrusion located adjacent the recess and wherein the protrusion deforms the mounting rail into the recess.

19. A mounting system for mounting an intramedullary bone attachment component to an artificial joint component comprising:
an artificial joint component having a first side with a bone attachment surface and a second side with an articulation surface;
a first mounting rail attached to the first side of the artificial joint component, wherein the first mounting rail defines a joint component mounting surface;
a mounting boss that is attachable to the first mounting rail, wherein the mounting boss comprises a first mounting surface which is attachable to the joint component mounting surface, and wherein the mounting boss comprises a second mounting surface which is attachable to an intramedullary bone attachment component to thereby attach the intramedullary bone attachment component to the artificial joint component;
a bolt comprising a threaded shank, a fastening head that does not engage a drive tool, and a drive head which is attached to the fastening head and which is formed with a drive surface that engages a drive tool, wherein the bolt passes through the mounting boss and attaches the mounting boss to the joint component, and wherein the drive head separates from the fastening head when a predetermined fastening torque is reached to inhibit removal of the bolt.

20. The mounting system of claim 19, further comprising an intramedullary bone attachment member that engages the second mounting surface to thereby attach the intramedullary bone attachment member to the mounting boss, and wherein the bolt drive head prevents attachment of the bone attachment member to the mounting boss until the bolt drive head is separated from the bolt.

21. The mounting system of claim 20, wherein the second mounting surface comprises a male tapered section that receives a female tapered section of an intramedullary bone attachment sleeve to thereby attach the bone ingrowth sleeve to the mounting boss, and wherein the bone attachment sleeve comprises a projection located adjacent the fastening head which contacts the drive head and prevents installation of the sleeve until the drive head is removed from the bolt.

22. The mounting system of claim 19, wherein the mounting boss is attached to the first mounting rail at a boss attachment joint comprising a mounting feature that deforms the first mounting rail when the mounting boss is attached to the first mounting rail to thereby inhibit movement of the mounting boss relative to the first mounting rail.

23. The mounting system of claim 22, wherein the device further comprises a nut, wherein the mounting boss is attached to a first side of the first mounting rail, wherein the nut is attached to a second side of the first mounting rail, and wherein the first mounting rail is clamped between the nut and the mounting boss by the bolt, wherein at least one of the nut and the mounting boss comprises a recess, and wherein the first mounting rail is deformed into the recess by the nut and mounting boss.

24. The mounting system of claim 23, wherein at least one of the nut and the mounting boss comprises a projection, and wherein the first mounting rail is deformed into the recess by the projection when the mounting rail is clamped against the mounting boss.

25. The mounting system of claim 22, wherein the joint component comprises a second mounting rail, a mounting slot defined between the first mounting rail and the second mounting rail, and wherein the mounting feature comprises an anti-rotation tab that extends between the first mounting rail and the second mounting rail and wherein a length of the anti-rotation tab is greater than a distance between the first mounting rail and the second mounting rail such that the first and second mounting rails are deformed when the anti-rotation tab is inserted between the first and second mounting rails.
